(19) United States
(12) Patent Application Publication
(10) Pub. No.: US 2016/0138047 A1
(43) Pub. Date: May 19, 2016

(54) IMPROVED POLYNUCLEOTIDE SEQUENCES ENCODING TALE REPEATS

(71) Applicant: CELLECTIS, Paris (FR)

(72) Inventors: Philippe DUCHATEAU, Draveil (FR); Sylvain ARNOULD, Saint-Denis (FR); Julianne SMITH, Le Plessis-Robinson (FR)

(21) Appl. No.: 14/898,148

(22) PCT Filed: Jun. 13, 2014
(86) PCT No.: PCT/EP2014/062405

§ 371 Date: Dec. 14, 2015

(30) Foreign Application Priority Data
Jun. 14, 2013 (DK) PA 2013 70321

(51) Int. Cl. C12N 15/90; C12N 15/86; C12N 7/00; C12N 9/22; C07K 14/00

(52) U.S. Cl. CPC C12N 15/907; C12N 9/22; C07K 14/00; C12N 7/00; C12N 15/86; C07K 2319/80; C12Y 301/21004; C12N 2740/15043; C12N 2740/15023; A61K 48/00

(57) ABSTRACT

The present invention is in the field of the gene editing molecular tools. The present invention relates to rewritten nucleic acid sequences encoding repeated DNA recognition motifs of TALE (Transcription Activator-Like Effector) proteins. These nucleic acid sequences allow assembly and cloning of TALE repeats in any type of vectors, especially viral vectors. The invention thereby contributes to improving gene targeting in cells using TALE derived proteins, in particular for genetic regulation or modification. The present invention is particularly drawn to virus mediated transformation methods, by providing vectors, compositions and kits including said new nucleic acid sequences.

### FIELD OF THE INVENTION

The present invention is in the field of the gene editing molecular tools. The present invention relates to rewritten nucleic acid sequences encoding repeated DNA recognition motifs of TALE (Transcription Activator-Like Effector) proteins. These nucleic acid sequences allow assembly and cloning of TALE repeats in any type of vectors, especially viral vectors. The invention thereby contributes to improving gene targeting in cells using TALE derived proteins, in particular for genetic regulation or modification. The present invention is particularly drawn to virus mediated transformation methods, by providing vectors, compositions and kits including said new nucleic acid sequences.

### BACKGROUND OF THE INVENTION

Repetitive DNA motifs are abundant in the genomes of various species and have the capacity to adopt non-canonical DNA structures. Non-canonical DNA structures have been shown to cause mutations, such as deletions, expansions and translocations in both prokaryotes and eukaryotes (Zhao, Bacolla et al. 2010). For instance, directly repeat sequences in both viral genomes and vectors are reported to be unstable and are deleted during viral replication, thus altering viral expression (see for review, (Delviks-Frankenberry, Galli et al. 2011)).

DNA tandem repeats are found in several DNA binding domains, in particular in Transcription Activator Like type III Effectors (TALEs). TALEs engineered nucleic acid binding domains have been mainly developed from a family of proteins, AvrBs3, involved in the infection process by the plant pathogens Xanthomonas. These proteins are characterized by highly repetitive motifs of 33-35 amino acids differing essentially by their 12^{th} and 13^{th} amino acid positions. Each base pair in the DNA target is contacted by such repeat, with the specificity resulting from these two variable 12^{th} and 13^{th} amino acid positions (the so-called repeat variable dipeptide, RVD) (Boch, Scholze et al. 2009; Moscou and Bogdanove 2009; Christian, Cermak et al. 2010; Li, Huang et al. 2011). Engineered TALEs have been adapted for gene targeting by arraying TALE repeats with RVDs corresponding to the target sequence of choice and fusing the resultant array to various catalytic domains (TALE derived proteins)(see WO2013017950). TAL derived proteins can be used as specific tools for gene regulation or modification. Fusion of TALE with VP16, for instance, allows activating gene expression from a particular promoter and fusion of TALE with a nuclease domain allows creating specific rare-cutting endonucleases acting at a desired locus, either under monomeric form such as in the case of TALE-I-TevI fusions, or under heterodimeric form, such as in the case of TALE-FokI fusions.

Current TALE-delivery methods mainly rely on nucleic acid electroporation, microinjection or transfection methods based on chemical reagents.

Nevertheless, viral vectors are hardly used for gene targeting because TALE proteins are made of repeated motifs that are highly identical to each other. The repeated nucleic acid sequences that encode TALE proteins make them difficult to clone these sequences into viral vectors, and also extensive genetic rearrangements have been observed in the cells transformed with such lentiviral vectors, resulting presumably from deletions involving the TALE repeats (Holkers, Maggio et al. 2012). These adverse effects currently prevent the use viral vectors for introducing nucleic acid sequences encoding TALE proteins into cells for gene editing, especially when genome integrity is sought, like for instance, to perform gene and cell therapy.

This particularly limits the use of TALE proteins into primary cells, such as T-cells, because primary cells are generally not permissive towards classical gene transfer technologies. Viral based vectors, derived from lentiviruses, are the main efficient technology to achieve high expression of transgenes in these cells.

In order to overcome the above limitations, the inventors have designed codon-optimized ready frames for engineering TALE nucleic acid sequences having increased sequence variability and sequence stability, while maintaining function of the encoded functional protein.

The resulting, so-called “rewritten TALE nucleic acid sequences” can be delivered into the cells particularly efficiently through viral vector for various applications ranging from targeted nucleic acid cleavage to targeted gene regulation.

### SUMMARY OF THE INVENTION

In a general aspect, the present invention provides a set of nucleic acid sequences encoding TALE modular binding domains allowing encoding engineered TALE proteins, while maximizing the nucleic acid polymorphism and stability between the different TALE units. These new polynucleotide sequences can be translated into proteins that retain binding function and specificity towards selected nucleic acid base sequences.

According to the invention, the polynucleotides encoding TALE proteins can be partially or totally “rewritten”, especially in view of being more easily cloned and inserted into plasmid or viral vectors for cell transformation. Such polynucleotides avoid troubleshooting and drawbacks due to identical sequences being repeated several times in the TAL sequences engineered according to the prior art.

The resulting polynucleotides encoding sequence TAL specific binding domains may be fused to further polynucleotide sequences encoding appropriate catalytic domains for gene processing, in particular domains conferring nuclease or transcriptional activity to the TALE proteins, and/or to particular N- and/or C-terminal domains.

The polynucleotides of the invention can be used in methods for gene targeting, in particular as specific nuclease for gene editing, more particularly for developing therapy based on viral vectorization. They are recommended for their use into lentiviral vectors, especially for the transformation of primary cells for stable expression and for stable transformation.

The present invention encompasses the viral vectors including these polynucleotides, the resulting viral particles, the cells transduced and genetically engineered using such viral particles, as well as the therapeutic use thereof.

### DESCRIPTION OF THE FIGURES

FIG. 1: T7 Endonuclease I treated DNA products loaded in a 10% acrylamide gel—The treated DNA products originate from Jurkat cells (A) not transduced (B) transduced with a lentiviral vector encoding TRAC TALEN™ comprising AvrBs3 repeat sequences (C) transduced with a lentiviral vector encoding TRAC TALEN™ comprising the repeat sequences according to the present invention (D) electroporated with mRNA encoding TRAC TALEN™ comprising the repeat sequences according to the present invention. Arrows indicate the fragment resulting from the T7 activity indicative of the endonuclease activity of TRAC TALEN™.

### DETAILED DESCRIPTION OF THE INVENTION

Unless specifically defined herein, all technical and scientific terms used have the same meaning as commonly understood by a skilled artisan in the fields of gene therapy, biochemistry, genetics, and molecular biology.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N. Y., 1986).

### Rewritten TALE Nucleic Acid

The present invention relates to a set of nucleic acid sequences encoding transcription activator like effector (TALE), allowing maximizing the polymorphisms between each TALE repeat sequences and avoiding unstable repeat tandem sequences. These nucleic acid sequences are particularly useful for assembling polynucleotides encoding engineered TALE nucleic acid binding domains.

As previously stated, transcription Activator like Effector (TALE) DNA binding domain comprises a plurality of TALE repeat sequences, each repeat comprising a Repeat Variable Diresidue (RVD) specific to each nucleotide base of a TALE recognition site (see WO2011/072246). Each TALE repeat sequence of said TALE is made of 30 to 42 amino acids, more preferably 33 or 34 wherein two critical amino acids (the so-called repeat variable dipeptide, RVD) located at positions 12 and 13 mediates the recognition of one nucleotide of said TALE binding site sequence; equivalent two critical amino acids can be located at positions other than 12 and 13 particularly in TALE repeat sequence larger than 33 or 34 amino acids long. As non limiting example said TALE repeat sequence derived from AvrBs3 is SEQ ID NO: 1 to 4 encoding amino acid sequence SEQ ID NO: 5.

Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. By other amino acid residues is intended any of the twenty natural amino acid residues or unnatural amino acid derivatives.

Said TALE DNA binding domain comprises between 5 and 30 TALE repeat sequences. More preferably, said nucleic acid encoding TALE comprises between 8 and 20 TALE repeat sequences; again more preferably 10 TALE repeat sequences.

The inventors have rewritten the nucleic acid sequence encoding AvrBs3 derived TALE repeat domain (SEQ ID NO: 1 to 4 encoding SEQ ID NO: 5), to maximize polymorphisms between each TALE repeat sequences. The present invention relates to nucleic acid encoding transcription activator like effector (TALE) repeat sequence selected from the group consisting of: SEQ ID NO: 6 to SEQ ID NO: 35. More particularly, the present invention relates to a nucleic acid encoding a TALE which comprises at least one repeat sequence selected from the group consisting of: SEQ ID NO: 6 to SEQ ID NO: 35. Preferably, said nucleic acid encoding TALE comprises at least 2, 3, 4, 5, 7, 10, 12, 15, 20, 30 repeat sequences selected from the group consisting of SEQ ID NO: 6 to SEQ ID NO: 35.

In another embodiment, said TALE can comprise an additional single truncated TALE repeat sequence made of 20 amino acids located at the C-terminus of said set of TALE repeat sequences, i.e. an additional C-terminal half-TALE repeat sequence. AvrBs3 derived half-TALE repeat sequence (SEQ ID NO: 36 encoding SEQ ID NO: 37) has also been modified to avoid DNA tandem repeat motifs. Thus, the nucleic acid according to the present invention can further comprise a half-TALE repeat sequence SEQ ID NO: 38 to SEQ ID NO: 39.

In particular embodiment, the present invention relates to a polynucleotide encoding TALE DNA biding domain selected from the group consisting of: SEQ ID NO: 40 to SEQ ID NO: 41.

TALE may further comprise an N-terminal domain which can be responsible for the requirement of a first thymine base (T₀) of the targeted sequence and a C-terminal domain which can contain a nuclear localization signals (NLS).

In a preferred embodiment according to the method of the present invention, said additional N-terminal and C-terminal domains of TALE DNA binding domains are derived from natural TALE. In a more preferred embodiment said additional N-terminal and C-terminal domains of engineered core TALE scaffold are derived from natural TALE like AvrBs3, PthXo1, AvrHah1, PthA, Tal1c as non-limiting examples. In another more preferred embodiment, said additional N-terminal and/or said C-terminal domains are truncated forms of respective N-terminal and/or said C-terminal domains of natural TALE like AvrBs3, PthXo1, AvrHah1, PthA, Tal1c as non-limiting examples from which they are derived. The inventors on the basis of truncated AvrBs3 N- and C-terminal sequences (SEQ ID NO: 42 and SEQ ID NO: 43 encoding respectively SEQ ID NO: 44 and 45) have engineered C- and N-terminal TALE sequences to avoid DNA repeat motifs within the TALE DNA binding domain sequence (SEQ ID NO: 46 and SEQ ID NO:47).

In a preferred embodiment, said TALE polynucleotide according to the present invention can comprise N-terminal sequence SEQ ID NO: 46 and/or C-terminal sequence SEQ ID NO: 47.

In another embodiment, said TALE of the present invention comprises TALE like repeat sequences of different origins. In a preferred embodiment, said TALE comprises TALE like repeat sequences originating from different naturally occurring TAL effectors. In another preferred embodiment, internal structure of some TALE like repeat sequences of the TALE of the present invention are constituted by structures or sequences originated from different naturally occurring TAL effectors. In another embodiment, said TALE of the present invention comprises TALE like repeat sequences. TALE like repeat sequences have a sequence different from naturally occurring TALE repeat sequences but have the same function and/or global structure within said core scaffold of the present invention.

It is understood that said nucleic acid encoding TALE according to the present invention can also comprise single or plural additional nucleotide substitutions or nucleotide insertion or nucleotide deletion introduced by mutagenesis process well known in the art. It is also encompassed in the scope of the present invention variants from TALE polynucleotide, according to the present invention. By variants, it is intended a nucleic acid encoding a TALE-nuclease optimized to avoid unstable DNA repeat motifs. As non limiting example, by variant it is intended a rewritten TALE-nuclease polynucleotide which is not prone to extensive rearrangement when it is introduced into cells through viral vector, preferably lentiviral vectors. Are also encompassed in the scope of the present invention TALE nucleic acid sequences with high percentage of identity or high percentage of homology with sequences described in the present invention at nucleotidic levels. By high percentage of identity or high percentage of homology it is intended 70%, more preferably 75%, more preferably 80%, more preferably 85%, more preferably 90%, more preferably 95, more preferably 97%, more preferably 99% or any integer comprised between 70% and 99% of SEQ ID NO: 6 to 35; SEQ ID NO: 38 to 41; SEQ ID No: 46 to 47.

### New Polynucleotides Encoding TALE-Fusion Proteins

The present invention also relates to polynucleotide encoding a TALE DNA binding domain fused an additional catalytic domain sequence to process the target genetic sequence.

In particular embodiment, said polynucleotide according to the present invention encodes a TALE which comprise at least one peptidic linker to fuse said TALE DNA binding domain and said catalytic. In a preferred embodiment, said peptidic linker is flexible. In another preferred embodiment, said peptidic linker is structured.

In a particular embodiment, the catalytic domain can be a transcription activator or repressor (i.e. a transcription regulator), or a protein that interacts with or modifies other proteins implicated in DNA processing. Non-limiting examples of DNA processing activities of said catalytic domain include, for example, creating or modifying epigenetic regulatory elements, making site-specific insertions, deletions, or repairs in DNA, controlling gene expression, and modifying chromatin structure.

The catalytic domain fused to the TALE DNA binding domain can have a catalytical activity selected from the group consisting of nuclease activity, polymerase activity, kinase activity, phosphatase activity, methylase activity, topoisomerase activity, integrase activity, transposase activity, ligase activity, helicase activity, recombinase activity, exonuclease activity. In a preferred embodiment, said catalytic domain is an endonuclease and the resulting fusion protein is a TALE-nuclease.

In particular embodiment, a first and a second TALE-nuclease can function respectively as monomer to act together as a dimer to process, preferably cleave the genetic sequence. As a non-limiting example, the two monomers can recognize different adjacent nucleic acid target sequences and the two protein domains function as subdomains that need to interact in order to process the genetic sequence.

As non-limiting example, said endonuclease can be a type IIS FokI endonuclease domain (for instance SEQ ID NO: 48 encoding SEQ ID NO: 49), engineered FokI to avoid repeat motifs within TALE-fusion protein sequence (SEQ ID NO: 50) or functional variant thereof which functions independently of the DNA binding domain and induces nucleic acid double-stranded cleavage as a dimer (Li, Wu et al. 1992; Kim, Cha et al. 1996).

In another particular embodiment, said TALE-nuclease is a monomeric TALE-nuclease that does not require dimerization for specific recognition and cleavage (see WO2012/138927). As non limiting example, such monomeric TALE-nuclease comprises a TALE DNA binding domain fused to the catalytic domain of I-TevI or a variant thereof.

Amino acid sequence of FokI or I-TevI variants can be prepared by mutations in the DNA, which encodes the catalytic domain. Such variants include, for example, deletions from, or insertions or substitutions of, residues within the amino acid sequence. Any combination of deletion, insertion, and substitution may also be made to arrive at the final construct, provided that the final construct possesses the desired activity. Said nuclease domain of FokI or I-TevI variant according to the present invention comprises a fragment of a protein sequence having at least 80%, more preferably 90%, again more preferably 95% amino acid sequence identity with the protein sequence of FokI or I-TevI: SEQ ID NO: 49 to 51.

In a more specific embodiment, the invention relates to a polynucleotide encoding a TALE-nuclease comprising sequence selected from the group consisting of SEQ ID NO: 52 to SEQ ID NO: 54.

It is understood that TALE-nucleases according to the present invention can also comprise single or plural additional nucleotide substitutions or nucleotide insertion or nucleotide deletion introduced by mutagenesis process well known in the art. It is also encompassed in the scope of the present invention variants from TALE-nucleases, according to the present invention. By variants, it is intended a polynucleotide encoding a TALE-nuclease optimized to avoid unstable DNA repeat motifs. As non limiting example, by variant it is intended a rewritten TALE-nuclease polynucleotide which is not prone to extensive rearrangement when it is introduced into cells through viral vector, preferably lentiviral vectors. Are also encompassed in the scope of the present invention TALE-nuclease variants which present a sequence with high percentage of identity or high percentage of homology with sequences SEQ ID NO: 52 to SEQ ID NO: 54, at nucleotidic levels. By high percentage of identity or high percentage of homology it is intended 70%, more preferably 75%, more preferably 80%, more preferably 85%, more preferably 90%, more preferably 95, more preferably 97%, more preferably 99% or any integer comprised between 70% and 99%.

### Vectors, Viral Vectors and Viral Particles

In another aspect, the invention relates to vectors comprising rewritten polynucleotide encoding TALE or TALE-fusion protein according to the present invention. The terms “vector” or “vectors” refer to a nucleic acid molecule capable of transporting another nucleic acid to which it has been linked. A “vector” in the present invention includes, but is not limited to, a viral vector, a plasmid, a RNA vector or a linear or circular DNA or RNA molecule which may consist of a chromosomal, non-chromosomal, semi-synthetic or synthetic nucleic acid. Preferred vectors are viral vectors, more particularly lentiviral vectors.

“viral vector” refers to a nucleic acid construct which carries, and within certain embodiments, is capable of directing the expression of a nucleic acid molecule of interest. The lentiviral vector can include at least one transcriptional promoter/enhancer or locus defining element(s), or other elements which control gene expression by other means such as alternate splicing, nuclear RNA export, post-translational modification of messenger, or post-transcriptional modification of protein. Such vector constructs can also include a packaging signal, long terminal repeats (LTRs) or portion thereof, and positive and negative strand primer binding sites appropriate to the retrovirus used (if these are not already present in the retroviral vector). Optionally, the recombinant lentiviral vector may also include a signal which directs polyadenylation, selectable markers such as Neo, TK, hygromycin, phleomycin, histidinol, or DHFR, as well as one or more restriction sites and a translation termination sequence. By way of example, such vectors typically include a 5′ LTR, a tRNA binding site, a packaging signal, an origin of second strand DNA synthesis, and a 3′ LTR or a portion thereof. Viral vectors include retrovirus, adenovirus, parvovirus (e. g. adenoassociated viruses), coronavirus, negative strand RNA viruses such as orthomyxovirus (e. g., influenza virus), rhabdovirus (e. g., rabies and vesicular stomatitis virus), paramyxovirus (e. g. measles and Sendai), positive strand RNA viruses such as picornavirus and alphavirus, and double-stranded DNA viruses including adenovirus, herpesvirus (e. g., Herpes Simplex virus types 1 and 2, Epstein-Barr virus, cytomegalovirus), and poxvirus (e. g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). More preferably, the present invention relates to a viral vector, preferably a lentiviral vector which comprises polynucleotide encoding TALE or TALE-fusion protein as described above. Particularly, the present invention relates to a viral vector comprising polynucleotide selected from the group consisting of: SEQ ID NO: 6 to 35; SEQ ID NO: 38 to 41; SEQ ID NO: 46 to 47; and SEQ ID NO: 52 to 54. Any of these vectors can comprise one or more polynucleotide encoding TALE or TALE fusion proteins. As non limiting example, one vector can comprise two sequences encoding two TALE monomers which can recognize different adjacent nucleic acid target sequences and the two protein domains function as subdomains that need to interact in order to process the genetic sequence. One vector can also comprise two sequences encoding two monomeric TALEs which recognize and process two different nucleic acid target sequences.

“Viral particle” as utilized within the present invention refers to a virus which carries at least one gene of interest. The virus may also contain a selectable marker. For instance, HIV type 1 (HIV-1) based vector particles may be generated by co-expressing the virion packaging elements and the vector genome in a so-called producer cell, e.g. 293T human embryonic kidney cells. These cells may be transiently transfected with a number of plasmids. Typically from three to four plasmids are employed, but the number may be greater depending upon the degree to which the lentiviral components are broken up into separate units. Generally, one plasmid encodes the core and enzymatic components of the virion, derived from HIV-1. This plasmid is termed the packaging plasmid. Another plasmid encodes the envelope protein(s), most commonly the G protein of vesicular stomatitis virus (VSV G) because of its high stability and broad tropism. This plasmid may be termed the envelope expression plasmid. Yet another plasmid encodes the genome to be transferred to the target cell, that is, the vector itself, and is called the transfer vector. Recombinant viruses with titers of several millions of transducing units per milliliter (TU/ml) can be generated by this technique and variants thereof. After ultracentrifugation concentrated stocks of approximately 10⁹ TU/ml can be obtained. The lentivirus is capable of reverse transcribing its genetic material into DNA and incorporating this genetic material into a host cell's DNA upon infection. Lentiviral vector particles may have a lentiviral envelope, a non-lentiviral envelope (e.g., an ampho or VSV-G envelope), or a chimeric envelope. The present invention relates to a viral, preferably a lentiviral particle which comprises new polynucleotides encoding TALE or TALE-fusion protein as described above. Particularly, the present invention relates to a viral particle comprising nucleic acid sequence selected from the group consisting of: SEQ ID NO: 6 to 35; SEQ ID NO: 38 to 41; SEQ ID NO: 46 to 47; and SEQ ID NO: 52 to 54.

### Method for Genetic Sequence Engineering

In another aspect, the present invention also relates to a method using new polynucleotides encoding TALE or TALE-fusion according to the present invention for various applications ranging from targeted nucleic acid cleavage to targeted gene regulation.

Particularly, the present invention relates to a method for targeting a genetic sequence within a cell comprising:
(a) selecting a genetic sequence in a cell comprising a target sequence;
(b) introducing into the cell a polynucleotide encoding a TALE nucleic acid binding domain matching said target sequence, comprising at least one repeat encoded by a nucleic acid as described above;
(c) expressing said nucleic acid such as the TALE nucleic acid binding domain binds the target genetic sequence.

Any genetic sequence can be processed by the present methods. For example, the genetic sequence can be chromosomal, organelle sequences such as mitochondrial or choloroplast sequences, or the genetic sequence can be a plasmid or viral sequence. The term “processing” as used herein means that the sequence is considered modified simply by the binding of the polypeptide. The term “processing” as used herein means for example promoting transcription activation around said nucleic acid target sequence.

In a particular embodiment, said additional protein domain is a nuclease domain, more preferably, endonuclease domain and the present invention more particularly relates to a method for cleaving a genetic sequence within a cell. For instance, the endonuclease domain can be a FokI or I-TevI catalytic domain. Depending on the endonuclease domain that constitutes said TALE nuclease, cleavage in the nucleic acid target sequence can correspond to either a double-stranded break or a single-stranded break.

The cleavage caused by endonuclease is commonly repaired through non-homologous end joining (NHEJ). NHEJ comprises at least two different processes. Mechanisms involve rejoining of what remains of the two DNA ends through direct re-ligation (Critchlow and Jackson 1998) or via the so-called microhomology-mediated end joining (Ma, Kim et al. 2003). Repair via non-homologous end joining (NHEJ) often results in small insertions or deletions and can be used for the creation of specific gene knockouts.

The present invention relates to a method for targeting the genetic sequence in a cell by expressing a polynucleotide encoding a TALE DNA binding domain, preferably A TALE-nuclease according to the present invention that allows nucleic acid cleavage that will lead to the loss of genetic information and any NHEJ pathway will produce targeted mutagenesis. In a preferred embodiment, the present invention relates to a method for modifying the genetic sequence of a cell by generating at least one nucleic acid cleavage and a loss of genetic information around said nucleic acid target sequence thus preventing any scarless re-ligation by NHEJ. Said modification may be a deletion of the genetic material, insertion of nucleotides in the genetic material or a combination of both deletion and insertion of nucleotides.

Cells in which a cleavage-induced mutagenesis event, i.e a mutagenesis event consecutive to an NHEJ event, has occurred can be identified and/or selected by well-known method in the art. As a non-limiting example, deep-sequencing analysis can be generated from the targeted cell genome around the targeted locus. Insertion/deletion events (mutagenesis events) can be therefore detected. As another non-limiting example, assays based on T7 endonuclease that recognizes non-perfectly matched DNA can be used, to quantify from a locus specific PCR on genomic DNA from provided cells, mismatches between reannealed DNA strands coming from cleaved/non-cleaved DNA molecules.

The present invention also relates to a method for modifying genetic sequence further comprising the step of expressing an additional catalytic domain into the cell. In a particular embodiment, the present invention relates to a method to increase mutagenesis wherein said additional catalytic domain is a DNA end-processing enzyme (see WO2012058458). Non limiting examples of DNA end-processing enzymes include 5-3′ exonucleases, 3-5′ exonucleases, 5-3′ alkaline exonucleases, 5′ flap endonucleases, helicases, hosphatase, hydrolases and template-independent DNA polymerases. Non limiting examples of such catalytic domain comprise of a protein domain or catalytically active derivate of the protein domain seleced from the group consisting of hExol (EXO1_HUMAN), Yeast ExoI (EXO1_YEAST), E. coli ExoI, Human TREX2, Mouse TREX1, Human TREX1, Bovine TREX1, Rat TREX1, tdt (terminal deoxynucleotidyl transferase) Human DNA2, Yeast DNA2 (DNA2_YEAST). In a preferred embodiment, said additional catalytic domain has a 3′-5′-exonuclease activity, and in a more preferred embodiment, said additional catalytic domain has TREX exonuclease activity, more preferably TREX2 activity. In another preferred embodiment, said catalytic domain is encoded by a single chain TREX polypeptide. Said additional catalytic domain may be fused to the chimeric protein according to the invention optionally by a peptide linker.

Endonucleolytic breaks are known to stimulate the rate of homologous recombination. Therefore, in another particular embodiment, when a polynucleotide encoding a TALE-nuclease is expressed, the present invention relates to a method for inducing homologous gene targeting in the target genetic sequence further comprising providing to the cell an exogeneous nucleic acid comprising at least a sequence homologous to a portion of the target genetic sequence such that homologous recombination occurs between the genetic sequence and the exogeneous nucleic acid.

Preferably, said exogenous nucleic acid comprises two sequences homologous to portions or adjacent portions of said genetic sequence flanking a sequence to introduce in the nucleic acid target sequence. Particularly, said exogenous nucleic acid comprises first and second portions which are homologous to region 5′ and 3′ of the genetic sequence target, respectively. Preferably, homologous sequences of at least 50 bp, preferably more than 100 bp and more preferably more than 200 bp are used within said exogenous nucleic acid. Therefore, the exogenous nucleic acid is preferably from 200 bp to 6000 bp, more preferably from 1000 bp to 2000 bp. Said exogenous nucleic acid in these embodiments can also comprise a third portion positioned between the first and the second portion which comprises no homology with the regions 5′ and 3′ of the nucleic acid target sequence. Following cleavage of the nucleic acid target sequence, a homologous recombination event is stimulated between the genome containing the genetic sequence and the exogenous nucleic acid. Indeed, shared nucleic acid homologies are located in regions flanking upstream and downstream the site of the cleavage and the nucleic acid sequence to be introduced should be located between the two arms. Said exogenous sequence allows to introduce new genetic material into a cell, replace or repair genetic material into a cell.

Cells in which a homologous recombination event has occurred can be selected by methods well-known in the art. As a non-limiting example, PCR analysis using one oligonucleotide matching within the exogenous nucleic acid sequence and one oligonucleotide matching the genomic nucleic acid of cells outside said exogenous nucleic acid but close to the targeted locus can be performed. Therefore, cells, in which methods of the invention allowed a mutagenesis event or a homologous recombination event to occur, can be selected.

Methods for introducing a nucleic acid into bacteria, plants, fungi and animal cells are known in the art and including as non limiting examples stable transformation methods wherein the nucleic acid construct is integrated into the genome of the cell, transient transformation methods wherein the nucleic acid construct is not integrated into the genome of the cell and virus mediated methods. Said nucleic acid may be introduced into a cell by for example, recombinant viral vectors (e.g. retroviruses, adenoviruses), liposomes and the like. For example, transient transformation methods include for example microinjection, electroporation or particle bombardment. Said nucleic acid may be included in vectors, more particularly plasmids or virus, in view of being expressed in prokaryotic or eukaryotic cells. As non-limiting example, said polynucleotide can be introduced as a transgene encoded by a plasmidic vector; said plasmidic vector may contain a selection marker which allows to identify and/or select cells which received said vector by method well-known in the art. Said protein expression can be induced in selected cells and said TALE or TALE-fusion protein binds target genetic sequence in selected cells, thereby obtaining cells in which TALE or TALE-fusion protein binds a specific target genetic sequence. Cells in which said nucleic acid has been introduced is selected by a selection method well-known in the art.

In particular embodiment of the invention, the method of targeting genetic sequence comprises selecting a genetic sequence within a cell, introducing the polynucleotide encoding TALE or TALE-fusion protein as described above into the cell via a viral particle, and expressing said polynucleotide within the cell. In particular, the viral particle comprises the polynucleotide and said polynucleotide is introduced into the cell by contacting said cell with the viral particle under condition that permits infection.

### Method for Generating an Animal/a Plant

In another aspect, the present invention relates to a non-human transgenic animal comprising a rewritten polynucleotide encoding a TALE or a TALE-fusion protein according to the present invention.

Animals may be generated by introducing polynucleotide encoding TALE or TALE fusion protein into a cell or an embryo. In particular, the present invention relates to a method for generating an animal, comprising providing an eukaryotic cell comprising a target genetic sequence into which it is desired to introduce a genetic modification; generating a cleavage within the target genetic sequence by introducing a polynucleotide encoding a TALE, preferably a TALE-nuclease according to the present invention; and generating an animal from the cell or progeny thereof, in which cleavage has occurred. Typically, the embryo is a fertilized one cell stage embryo. Rewritten polynucleotide according to the invention may be introduced into the cell by any of the methods known in the art including micro injection into the nucleus or cytoplasm of the embryo. In a particular embodiment, the method for generating an animal, further comprise introducing an exogenous nucleic acid as desired. The exogenous nucleic acid can include for example a nucleic acid sequence that disrupts or replaces a gene after homologous recombination, a nucleic acid sequence that introduces a mutation into a gene after homologous recombination or a nucleic acid sequence that introduce a regulatory site after homologous recombination. The embryos are then cultures to develop an animal. In one aspect of the invention, an animal in which at least a target nucleic acid sequence of interest has been engineered is provided. For example, an engineered gene may become inactivated such that it is not transcribed or properly translated, or an alternate form of the gene is expressed. The animal may be homozygous or heterozygous for the engineered gene.

The present invention also relates to a transgenic plant comprising a rewritten polynucleotide encoding a TALE or a TALE-fusion protein according to the present invention. Plants may be generated by providing a plant cell comprising a target genetic sequence into which it is desired to introduce a genetic modification; generating a cleavage within the target genetic sequence by introducing rewritten polynucleotide encoding a TALE or a TALE-fusion protein according to the invention; and generating a plant from the cell or progeny thereof, in which cleavage has occurred. Progeny includes descendants of a particular plant or plant line. In a particular embodiment, the method for generating a plant, further comprise introducing an exogenous nucleic acid as desired. Said exogenous nucleic acid comprises a sequence homologous to at least a portion of the target genetic sequence, such that homologous recombination occurs between said exogenous nucleic acid and the target nucleic acid sequence in the cell or progeny thereof. Plant cells produced using methods can be grown to generate plants having in their genome a modified target genetic sequence. Seeds from such plants can be used to generate plants having a phenotype such as, for example, an altered growth characteristic, altered appearance, or altered compositions with respect to unmodified plants.

### Therapeutic Application

The method disclosed herein can have a variety of applications. In one embodiment, the method can be used for clinical or therapeutic applications. The method can be used to repair or correct disease-causing genes, as for example a single nucleotide change in sickle-cell disease. The method can be used to correct splice junction mutations, deletions, insertions, and the like in other genes or chromosomal sequences that play a role in a particular disease or disease state.

From the above, the polypeptides according to the invention can be used as a medicament, especially for modulating, activating or inhibiting gene transcription, at the promoter level or through their catalytic domains.

Rewritten polynucleotide encoding TALE or TALE-fusion protein according to the present invention can be used for the treatment of a genetic disease to correct a mutation at a specific locus or to inactivate a gene the expression of which is deleterious. Such proteins can also be used to genetically modify iPS or primary cells, for instance T-cells, in view of injected such cells into a patient for treating a disease or infection. Such cell therapy schemes are more particularly developed for treating cancer, viral infection such as caused by CMV or HIV or self-immune diseases.

Viral particles comprising one or more polynucleotides encoding TALE or TALE-fusion protein can be used as a medicament or for gene therapy, by administrating said viral particle directly into the subject, or by transforming cells with said viral particle and administrating cells into the subject.

Still according to the present invention, immune cells from donors or patients, such as T-cells can be transduced using a viral vector as previously described, in order to genetically engineer such cells and enhance or redirect their cytotoxic activity towards certain types of malignant or pathological cells. For instance, by using the lentiviral vectors according to the invention encoding TAL-nucleases directed against T-cells receptors (TCR) T-cells can be turned into allogeneic T-cells. These cells may be infused to patients in order to reinforce their immune response against malignancies or infections. The production of allogeneic T-cells for immunotherapy using specific endonucleases, such as TALEN™ is described in WO 2013176915, which is incorporated by reference. Thus, the present invention more particularly relate to a method for producing allogeneic T-cells comprising one or several of the steps of:
i) preparing a viral vector encoding a TAL nuclease targeting a TCR gene by using at least one or several of the polynucleotides sequences according to the invention,
ii) transducing T-cells form patient or donor using said viral vector,
iii) separating a population of transduced T-cells in which more than 50%, preferably 60% and more preferably 70% of the T cells are inactivated for said TCR gene.
iv) Formulating said population of T-cells into a therapeutic composition.

### Modified Cells, Kits and Compositions

It is also encompassed in the scope of the present invention an isolated cell comprising a rewritten polynucleotide encoding a TALE or a TALE-fusion protein according to the present invention.

The rewritten polynucleotide of the invention is useful to engineer genomes and to reprogram cells, especially induced pluripotent stem cells, embryonic stem cells and primary cells, such as T-cells.

Cells can be modified by the method of the present invention to provide cell line models to produce, express, quantify, detect, study a gene or a protein of interest; these models can also be used to screen biologically active molecules of interest in research and production and various fields such as chemical, biofuels, therapeutics and agronomy as non-limiting examples.

The present invention also relates to a kit comprising at least a rewritten nucleic acid or a vector, preferably a viral vector according to the present invention and instructions for use said kit.

The present invention also relates to a composition comprising at least a rewritten polynucleotide or vector according to the present invention and a carrier. More preferably, is a pharmaceutical composition comprising such nucleic acid or vector and a pharmaceutically active carrier. For purposes of therapy, the rewritten polynucleotide according to the present invention and a pharmaceutically acceptable excipient are administered in a therapeutically effective amount. Such a combination is said to be administered in a “therapeutically effective amount” if the amount administered is physiologically significant. An agent is physiologically significant if its presence results in a detectable change in the physiology of the recipient. In the present context, an agent is physiologically significant if its presence results in a decrease in the severity of one or more symptoms of the targeted disease and in a genome correction of the lesion or abnormality.

### Other Definitions

In the description above, a number of terms are used extensively. The following definitions are provided to facilitate understanding of the present embodiments.

Amino acid residues in a polypeptide sequence are designated herein according to the one-letter code, in which, for example, Q means Gln or Glutamine residue, R means Arg or Arginine residue and D means Asp or Aspartic acid residue.

Amino acid substitution means the replacement of one amino acid residue with another, for instance the replacement of an Arginine residue with a Glutamine residue in a peptide sequence is an amino acid substitution.

Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.

As used herein, “nucleic acid”, polynucleotide” or “genetic sequence” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.

By “cells” is intended any prokaryotic or eukaryotic living cells, cell lines derived from these organisms for in vitro cultures, primary cells from animal or plant origin. By “primary cell” or “primary cells” are intended cells taken directly from living tissue (i.e. biopsy material) and established for growth in vitro, that have undergone very few population doublings and are therefore more representative of the main functional components and characteristics of tissues from which they are derived from, in comparison to continuous tumorigenic or artificially immortalized cell lines. These cells thus represent a more valuable model to the in vivo state they refer to. Additionally primary cells can be isolated and used ex vivo for reintroduction into the subject to be treated following treatment with rewritten nucleic acid encoding TALE or TALE fusion protein according to the present invention. Suitable primary cells include peripherical blood mononuclear cells (PBMC), and other blood subsets such as, but not limited to, CD4⁺ T cells or CD8⁺ T cells. Suitable cells also include stem cells such as, by way of example, embryonic stem cells, induced pluripotent stem cells, hematopoietic stem cells, neuronal stem cells, muscle stem cells, and skin stem cells. In the frame of the present invention, “eukaryotic cells” can refer to a fungal, plant, algal or animal cell or a cell line derived from the organisms listed below and established for in vitro culture.

In the present invention, the cell is preferably a plant cell, a mammalian cell, a fish cell, an insect cell or cell lines derived from these organisms for in vitro cultures or primary cells taken directly from living tissue and established for in vitro culture. As non limiting examples cell lines can be selected from the group consisting of CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; Molt 4 cells.

As non limiting example cells according to the present invention can be stem cells, embryonic stem cells and induced Pluripotent Stem cells (iPS). In particular, the cells can be embryonic stem cells not obtained by destruction of human embryos.

By “homologous sequence” it is meant a nucleic acid sequence with enough identity to another one to lead to homologous recombination between sequences, more particularly having at least 80% identity, preferably at least 90% identity and more preferably at least 95%, and even more preferably 98% identity. “Identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting.

Having generally described this invention, a further understanding can be obtained by reference to certain specific examples, which are provided herein for purposes of illustration only, and are not intended to be limiting unless otherwise specified.

### Examples

### A. Rewriting Process

Codon and secondary structure optimization were performed on AvrBs3 derived TALE sequence to optimize sequence diversity and stability within the TALE repeat array sequence using codon usage database. Nucleic acid sequence encoding specific amino acid sequence was rewritten with overall codon usage by taking into account the RNA secondary structure and/or relative t-RNA frequency, until obtaining a polynucleotide sequence without repeated motifs. Two half-TALE-nucleases TRAC01-R and TRAC01-L targeting TCR alpha gene were optimized in such a way (SEQ ID NO: 55 to SEQ ID NO: 56) into three partially or totally rewritten TALE-nucleases (SEQ ID NO: 52 to SEQ ID NO: 54) comprising TALE DNA binding domain (SEQ ID NO: 40 and SEQ ID NO: 41) to be cloned into plasmid vectors and tested for nuclease activity towards TCRalpha gene

### B. pLV Plasmid Manufacturing Process

Rewritten TRAC01 TALE-nuclease sequences (SEQ ID NO: 52 to SEQ ID NO: 54) were cloned into a pLV.EF1 long lentiviral expression plasmid (Vectalys SAS, Canal Biotech II, 31400 Toulouse, France) under the control of the EF1alpha long promoter by restriction and ligation using standard biological tools. Candidate clones were selected and screened by colony PCR. Resulting screened constructs were then sequenced.

### C. rLV Manufacturing Process

Viral derived particles were produced by tri-transfection into 293T cells by using standard phosphate calcium procedures. 24 hours after transfection, cells were washed with medium. Viral supernatants were collected 24 hours later, filtered through 0.45 μm filters and submitted to ultrafiltration to purify and concentrate the vector batch. Titers were estimated by qPCR 72 hours after transduction of HCT116 cells by serial dilutions of viral supernatants.

The viral titers above correspond to the number of viral DNA copies generated 3 days after transduction of 1E5 HCT116 cells with one milliliter of viral supernatant in presence of 8 μg/ml polybrene.

### D. rLV Transduction Process

Jurkat cells (1×10⁵) were added in 96-well plate in 1004 of PBS 2% Fetal Bovine Serum (FBS) plus 8 μg/mL polybrene for 1 h at 37° C. Cells were successively washed with PBS-2% FBS then with PBS. Lentiviral supernatant was added to cells and cells are incubated for 3 to 4 h at 37° C. in an atmosphere of 5% CO₂. Cells were then transferred to 24-well plates with 5004 RPMI, 10% FBS, 1% P/S, and 10 mM Hepes (cRPMI) and cultured overnight. After 48 h, a fraction of cells is analyzed by flow cytometry to evaluate the transduction efficiency. Cell number is determined using a Countess Automated Cell Counter.

### E. Activity of Rewritten TRAC-TALE-Nuclease

The double stranded cleavage generated by TALE-nucleases in TRAC coding sequences is repaired in live cells by non homologous end joining (NHEJ), which is an error-prone mechanism. Activity of TALE-nucleases in live cells is measured by the frequency of insertions or deletions at the genomic locus targeted. Several days after transduction, genomic DNA was isolated from transduced cells and locus specific PCRs were performed. PCR products were sequenced by a 454 sequencing system (454 Life Sciences). Approximately 10,000 sequences were obtained per PCR product and then analyzed for the presence of site-specific insertion or deletion events.

### F. Activity of Rewritten TRAC-TALE-Nuclease (T7 Endonuclease Assay)

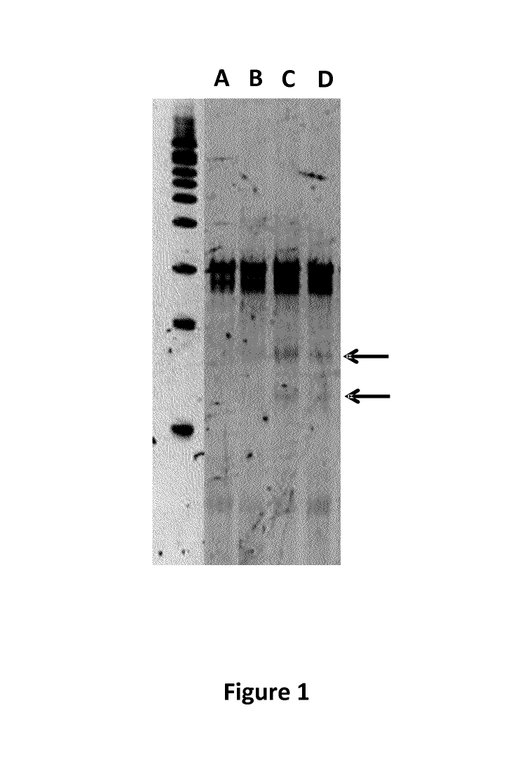

The double stranded cleavage generated by TALE-nucleases in TRAC coding sequences is repaired in live cells by non-homologous end joining (NHEJ), which is an error-prone mechanism. In order to evidence these mutagenic events (small deletions and insertions, Indels) induced by the TALE-nucleases, T7 endonuclease was performed on the Jurkat cells respectively transduced above in D. paragraph with respectively the lentiviral vectors comprising the original AvrBs3 sequences (B) and the rewritten sequences (C). The mutagenic events were monitored by SDS-PAGE by performing (i) a whole cell population genomic DNA extraction (ii) a specific PCR locus around the TRAC locus on the extracted genomic DNA using oligos TRACf 5′-CCATCTCATCCCTGCGTGTCTCCGACTCAGTAGAGACGAGTTGGCCAAGATTGATAGCTTGTGCC-3′ (SEQ ID NO: 57) and TRACr 5′-CCTATCCCCTGTGTGCCTTGGCAGTCTCAGAAGTCAGATTTGTTGCTCCAGGCCA-3′ (SEQ ID NO: 58) and (iii) an enzymatic T7 Endonuclease assay based on the protocol described by Joung and Colleagues (Reyon, D. et al., 2012): 50 ng of the purified PCR amplicon (19 μl total volume) was denatured and reannealed in an annealing buffer (Tris 10 mM, EDTA 1 mM, NaCl 100 mM) using a thermocycler with the following protocol: 95° C., 10 min; 95-85° C. at −3° C./s; 85-25° C. at −0.3° C./s. The reannealed PCR products (20 μl) were digested with 0.5 μl of T7 Endonuclease I (New England Biolabs, cat. M0302L) in NEB2 buffer for 15 minutes at 37° C. 10 μl of the T7 Endonuclease I treated DNA products are loaded in a 10% acrylamide gel and DNA products are revealed with SYBRgreen (Sigma Aldrich, cat. S9430-1ML). The DNA products were compared with those obtained from non-transduced cells (negative control A) and from Jurkat cells transformed with mRNA encoding TRAC TALEN comprising the same rewritten sequences (D). The resulting gel is displayed in FIG. 1. The two fragments resulting from the T7 digest detectable in C and D indicate that the rewritten sequences have enabled the expression of TAL nucleases that were able to cut TCRalpha gene (TRAC). This shows that the TALEN™ encoded by the polynucleotide sequences according to the invention can be cloned into a lentiviral vector allowing the efficient expression of a TAL polypeptide product with specific binding properties and endonuclease activity.

### REFERENCES

Boch, J., H. Scholze, et al. (2009). “Breaking the code of DNA binding specificity of TAL-type III effectors.” Science 326(5959): 1509-12.
Christian, M., T. Cermak, et al. (2010). “Targeting DNA double-strand breaks with TAL effector nucleases.” Genetics 186(2): 757-61.
Critchlow, S. E. and S. P. Jackson (1998). “DNA end-joining: from yeast to man.” Trends Biochem Sci 23(10): 394-8.
Delviks-Frankenberry, K., A. Galli, et al. (2011). “Mechanisms and factors that influence high frequency retroviral recombination.” Viruses 3(9): 1650-80.
Holkers, M., I. Maggio, et al. (2012). “Differential integrity of TALE nuclease genes following adenoviral and lentiviral vector gene transfer into human cells.” Nucleic Acids Res 41(5): e63.
Kim, Y. G., J. Cha, et al. (1996). “Hybrid restriction enzymes: zinc finger fusions to Fok I cleavage domain.” Proc Natl Acad Sci USA 93(3): 1156-60.
Li, L., L. P. Wu, et al. (1992). “Functional domains in Fok I restriction endonuclease.” Proc Natl Acad Sci USA 89(10): 4275-9.
Li, T., S. Huang, et al. (2011). “TAL nucleases (TALNs): hybrid proteins composed of TAL effectors and FokI DNA-cleavage domain.” Nucleic Acids Res 39(1): 359-72.
Ma, J. L., E. M. Kim, et al. (2003). “Yeast Mre11 and Rad1 proteins define a Ku-independent mechanism to repair double-strand breaks lacking overlapping end sequences.” Mol Cell Biol 23(23): 8820-8.
Moscou, M. J. and A. J. Bogdanove (2009). “A simple cipher governs DNA recognition by TAL effectors.” Science 326(5959): 1501.
Reyon, D., Tsai, S. Q., Khayter, C., Foden, J. A., Sander, J. D., and Joung, J. K. (2012). “FLASH assembly of TALENs for high-throughput genome editing”. Nat Biotechnol.
Zhao, J., A. Bacolla, et al. (2010). “Non-B DNA structure-induced genetic instability and evolution.” Cell Mol Life Sci 67(1): 43-62.

1. A nucleic acid encoding a TALE repeat, wherein said nucleic acid has at least 95% sequence identity with any of the sequences selected from the group consisting of SEQ ID NO: 6 to SEQ ID NO: 35 and SEQ ID NO: 38 to SEQ ID NO: 39.

2. A polynucleotide encoding a TALE DNA binding domain comprising at least one nucleic acid of claim 1.

3. A polynucleotide according to claim 2 comprising, at least two, preferably at least 10, more preferably at least 12 nucleic acid of claim 1, having a sequence different from each other.

4. A polynucleotide according to claim 2 or 3, wherein it comprises a sequence having at least 95% identity with SEQ ID NO: 40 or SEQ ID NO: 41.

5. The polynucleotide of any one of claims 2 to 4, further comprising an N-terminal sequence.

6. The polynucleotide of claim 5, wherein said N-terminal sequence is SEQ ID NO: 46.

7. The polynucleotide according to any one of claims 2 to 6 comprising a C-terminal sequence.

8. The polynucleotide of claim 7, wherein said C-terminal sequence is SEQ ID NO: 47.

9. The polynucleotide according to any one of claims 2 to 8, wherein said TALE DNA binding domain is fused to a catalytic domain sequence.

10. The polynucleotide of claim 9, wherein the catalytic domain is a transcriptional activator or repressor.

11. The polynucleotide of claim 9, wherein the catalytic domain is an endonuclease domain.

12. The polynucleotide of claim 11, wherein the endonuclease domain is FokI.

13. The polynucleotide of claim 12, wherein the FokI nucleotide sequence is SEQ ID NO: 50.

14. The polynucleotide of claim 12 selected from the group consisting of: SEQ ID NO: 52 to SEQ ID NO: 54.

15. A viral vector comprising the polynucleotide according to any one of claims 2 to 14.

16. A lentiviral vector comprising the polynucleotide according to any one of claims 2 to 14.

17. A viral particle obtainable from a viral vector according to claim 15 or 16.

18. A method for targeting a genetic sequence within a cell comprising:
(a) selecting a genetic sequence in a cell comprising a target sequence;
(b) introducing into the cell a polynucleotide encoding a TALE nucleic acid binding domain matching said target sequence, comprising at least one repeat encoded by a nucleic acid having at least 95% sequence identity with any of the sequences selected from the group consisting of SEQ ID NO: 6 to SEQ ID NO: 35 and SEQ ID NO: 38 to SEQ ID NO: 39.
(c) Expressing said nucleic acid within the cell such as the TALE nucleic acid binding domain binds the target genetic sequence.

19. The method of claim 18, wherein said polynucleotide of step b) is according to any one of claims 2 to 17.

20. The method according to any one of claim 18 or 19, wherein said polynucleotide encoding said TALE nucleic acid binding domain is fused to a domain with nuclease activity for cleaving said genetic sequence within the cell

21. The method according to any one of claims 18 to 20 wherein said polynucleotide is introduced into the cell via a viral vector.

22. The method of claim 21, wherein said nucleic acid is introduced into the cell by contacting a viral particle which comprises said nucleic acid with the cell.

23. The method according to claims 18 to 22 comprising introducing an exogenous nucleic acid sequence into the cell comprising at least one sequence homologous to at least a portion of the genetic sequence in order to perform homologous recombination at the site of cleavage.

24. The viral particle according to claim 17 for use as a medicament.

25. The viral particle according to claim 24 for use in gene therapy.

26. A cell transformed with a polynucleotide according to any one of the claims 1 to 14.
